(19) United States
(12) Patent Application Publication
(10) Pub. No.: US 2012/0100600 A1
(43) Pub. Date: Apr. 26, 2012

(54) APPARATUS FOR POLYNUCLEOTIDE DETECTION AND QUANTITATION

(75) Inventor: Vladimir I. Slepnev, Cincinnati, OH (US)

(73) Assignee: PRIMERADX, INC., Mansfield, MA (US)

(21) Appl. No.: 13/312,584

(22) Filed: Dec. 6, 2011

Related U.S. Application Data
(63) Continuation of application No. 10/464,941, filed on Jun. 19, 2003.
(60) Provisional application No. 60/390,269, filed on Jun. 20, 2002.

(51) Int. Cl. C12M 1/40

(52) U.S. Cl. 435/287.2

(57) ABSTRACT

An apparatus for expression profiling analysis, subjecting biological materials to polynucleotide extraction, amplification and analysis. The apparatus include an amplification device which permits the amplification of polynucleotides and an analysis device which quantifies the amount of the amplified polynucleotide products. The amplification device of the apparatus may further permit polynucleotide extraction to prepare the template for amplification, or sequence identification of a quantified polynucleotide product. A fraction collector may be included in the apparatus to collect a qualified polynucleotide product before its sequence is identified. The analysis device may further permit data generation, or alternatively, data can be generated by a separate data generation device provided with the apparatus. The devices within the apparatus are connected by connecting means which permit the transfer of a fluid or a signal for amplification and analysis.

### RELATED APPLICATIONS

This application claims priority to U.S. Provisional Application with a Ser. No. 60/390,269, filed Jun. 20, 2002, the entirety is incorporated herein by reference.

### FIELD OF THE INVENTION

The present invention relates to an automated apparatus to be used for the detection and quantitation of polynucleotides.

### BACKGROUND

The introduction of genomics has been instrumental in accelerating the pace of drug discovery. The genomic technologies have proved their value in finding novel drug targets. Further improvement in this area will provide more efficient tools resulting in faster and more cost efficient development of potential drugs.

The drug discovery process includes several steps: the identification of a potential biochemical target associated with disease, screening for active compounds and further chemical design, preclinical tests, and finally clinical trials. The efficiency of this process is still far from perfect: it is estimated that about 75% of money spent in the research and development process funds went to failed projects. Moreover, the later in the product development a failure occurs, the bigger are the losses associated with this project. Therefore, there is a need for early elimination of future failures to considerably cut costs of the whole drug development process. Thus, the quality of the original molecular target becomes a decisive factor for cost-effective drug development.

One approach that promises to impact on the process of target identification and validation is transcription profiling. This method compares expression of genes under specific conditions: for example, between disease and normal cells, between control and drug-treated cells or between cells responding to treatment and those resistant to it. The information generated by this approach may directly identify specific genes to be targeted by a therapy, and, importantly, reveals biochemical pathways involved in disease and treatment. In brief, transcription profiling not only provides biochemical targets, but at the same time, a way to assess the quality of these targets. Moreover, in combination with cell-based screening, transcription profiling is positioned to dramatically change the field of drug discovery. Historically, screening for a potential drug was successfully performed using phenotypic change as a marker in functional cellular system. For example, growth of tumor cells in culture was monitored to identify anticancer drugs. Similarly, bacterial viability was used in assays aimed at identifying antibiotic compounds. Such screens were typically conducted without prior knowledge of the targeted biochemical pathway. In fact, the identified effective compounds revealed such pathways and pointed out the true molecular target, enabling subsequent rational design of the next generations of drugs.

Modern tools of transcription profiling can be used to design novel screening methods that will utilize gene expression in place of phenotypic changes to assess effectiveness of a drug. For example, these methods are described in U.S. Pat. Nos. 5,262,311; 5,665,547; 5,599,672; 5,580,726; 6,045,988; and 5,994,076, as well as Luehrsen et al. (1997, Biotechniques, 22:168-74; Liang and Pardee (1998, Mol. Biotechnol. 10:261-7). Such approaches will be invaluable for drug discovery in the field of central nervous system (CNS) disorders such as dementia, mild cognitive impairment, depression, etc., where phenotypic screening is inapplicable, but the desired transcription profile can be readily established and linked to particular disorders. The identified effective compounds will reveal the underlying molecular processes. In addition, this method can be instrumental for development of improved versions of existing drugs, which act at several biochemical targets at the same time to generate the desired pharmacological effect. In such cases, the change in the transcriptional response may be a better marker for drug action than selection based on optimization of binding to multiple targets.

Prior to the present invention, the most advanced method of transcription profiling is based on technology using DNA microarrays, for example, as reviewed in Greenberg, 2001 Neurology 57:755-61; Wu, 2001, J. Pathol. 195:53-65; Dhiman et al., 2001, Vaccine 20:22-30; Bier et al., 2001 Fresenius J Anal Chem. 371:151-6; Mills et al., 2001, Nat Cell Biol. 3:E175-8; and as described in U.S. Pat. Nos. 5,593,839; 5,837,832; 5,856,101; 6,203,989; 6,271,957; and 6,287,778. DNA microarray is a method which performs simultaneous comparison of the expression of several thousand genes in a given sample by assessing hybridization of the labeled polynucleotide samples, obtained by reverse transcription of mRNAs, to the DNA molecules attached to the surface of the test array. While the prior art provides valuable information about transcriptional changes, it is far from perfect and not without problems and drawbacks.

First, this technology is limited to the pool of genes presented in the microarray. The current printing methods allows placement of 10,000-15,000 genes on a single chip, which is essentially a number of genes expressed in a particular cell type. Given the diversity of cell types, it requires development of specific arrays liar specific cell types. While theoretically possible, this task is nearly impossible to achieve, since it requires knowledge of the gene pool expressed in these cells prior to microarray manufacturing.

Moreover, the number of transcripts in a tissue sample is even higher than in a cellular sample and will exceed the capacity of the microarray. In addition, some changes in gene expression result from alternative splicing, which further increases the number of transcripts that need to be assessed. The only possibility to overcome these difficulties will be to develop multiple arrays that will cover the entire genome, including alternatively spliced genes. This approach will significantly increase the cost of a single experiment and will require a large biological sample, perhaps larger than is reasonably available.

Second, prior art DNA microarrays do not provide quantitatively accurate data, and observed changes in gene expression must be confirmed by an independent method (for example, quantitative polymerase chain reaction (Q-PCR).

Finally, rare transcripts, which may be of particular interest, can not be detected by microarrays using prior art detection techniques.

Capillary electrophoresis has been used to quantitatively detect gene expression. Rajevic at el. (2001, Pflugers Arch. 442(6 Suppl 1):R190-2) discloses a method for detecting differential expression of oncogenes by using seven pairs of primers for detecting the differences in expression of a number of oncogenes simultaneously. Sense primers were 5′ end-labelled with a fluorescent dye. Multiplex fluorescent RT-PCR results were analyzed by capillary electrophoresis on ABI-PRISM 310 Genetic Analyzer. Borson et al. (1998, Biotechniques 25:130-7) describes a strategy for dependable quantification of low-abundance mRNA transcripts based on quantitative competitive reverse transcription PCR (QC-RT-PCR) coupled to capillary electrophoresis (CE) for rapid separation and detection of products. George et al., (1997, J Chromatogr B Biomed Sci Appl 695:93-102) describes the application of a capillary electrophoresis system (ABI 310) to the identification of fluorescent differential display generated EST patterns. Odin et al. (1999, J Chromatogr B Biomed Sci Appl 734:47-53) describes an automated capillary gel electrophoresis with multicolor detection for separation and quantification of PCR-amplified cDNA.

Separate devices are available for PCR amplification and CE. For example, PCR machines are commercially available from Applied Biosystems (Foster City, Calif.), Bio-Rad (Hercules, Calif.), Eppendorf (Westbury, N.Y.), Roche (Indianapolis, Ind.). CE apparatuses are commercially available from Applied Biosystems (Foster City, Calif.), Beckman Coulter (Fullerton, Calif.), and Spectrumedix Corporation (State College, Pa.).

U.S. Pat. No. 6,126,804 discloses an instrument for field identification of micro-organisms and DNA fragments using a small and disposable device containing integrated polymerase chain reaction (PCR) enzymatic reaction wells, attached capillary electrophoresis (CE) channels, detectors, and read-out all on/in a small hand-held package. However, this instrument is specifically designed for field use. Further, no prior art device offers a simple, sensitive apparatus for quantitative detection of gene expression profile in one or more samples.

To overcome these limitations, there is a need in the art to develop alternative apparatus to perform transcription profiling that will: (1) not require prior knowledge of the sequences of the expressed gene pool before the assay, but by itself will provide this information during/after the assay; (2) measure quantitative changes in the level of expressed transcripts; (3) detect expression of rare genes; and (4) be automated. There is a need in the art for a simple, sensitive apparatus for quantitative detection of gene expression profile in one or more samples.

### SUMMARY OF THE INVENTION

The present invention provides an apparatus for expression profiling, comprising an amplification device which amplifies a polynucleotide in a reaction mixture to generate an amplified product; and an analysis device connected to the amplification device by a first connecting means which permits an aliquot of the reaction mixture to transfer from the amplification device to the analysis device which detects and quantifies the amplified product, where the first connecting means is a robotic arm.

In one embodiment, the apparatus further comprises a polynucleotide extraction device connected to the amplification device by a second connecting means which permits an extracted polynucleotide sample to transfer from the polynucleotide extraction device to the amplification device.

In another embodiment, the apparatus further comprises a fraction collector device.

In a preferred embodiment, the fraction collector device is connected to the analysis device by a fourth connecting means which permits the collection of a quantified product.

In another embodiment, the apparatus further comprises a sequence identifier which identifies the sequence of a quantified product, where the sequence identifier is connected to the analysis device by a fifth connecting means which permits a quantified product to transfer from the analysis device to the sequence identifier.

In another embodiment, the apparatus further comprises a sequence identifier which identifies the sequence of a quantified product, where the sequence identifier is connected to the fraction collector device by a fifth connecting means which permits a collected product to transfer from the fraction collector device to the sequence identifier.

The present invention also provides an apparatus for expression profiling comprising an amplification device which amplifies a polynucleotide in a reaction mixture to generate an amplified product; an analysis device connected to the amplification device by a first connecting means which permits an aliquot of the reaction mixture to transfer from the amplification device to the analysis device which detects and quantifies the amplified product; and a polynucleotide extraction device connected to the amplification device by a second connecting means which permits an extracted polynucleotide sample to transfer from the polynucleotide extraction device to the amplification device.

In one embodiment, the apparatus further comprises a fraction collector device.

In a preferred embodiment, the fraction collector is connected to the analysis device by a fourth connecting means which permits the collection of a quantified product.

In another embodiment, the apparatus further comprises a sequence identifier which identifies the sequence of a quantified product, where the sequence identifier is connected to the analysis device by a fifth connecting means which permits a quantified product to transfer from the analysis device to the sequence identifier.

In another embodiment, the apparatus further comprises a sequence identifier which identifies the sequence of a quantified product, where the sequence identifier is connected to the fraction collector device by a fifth connecting means which permits a collected product to transfer from the fraction collector device to the sequence identifier.

The invention provides an apparatus for expression profiling comprising an amplification device which amplifies a polynucleotide in a reaction mixture to generate an amplified product; an analysis device connected to the amplification device by a first connecting means which permits an aliquot of the reaction mixture to transfer from the amplification device to the analysis device which detects and quantifies the amplified product; and a data generating device connected to the analysis device by a third connecting means which permits a signal to transfer from the analysis device to the data generating device.

In one embodiment, the apparatus further comprises a polynucleotide extraction device connected to the amplification device by a second connecting means which permits an extracted polynucleotide sample to transfer from the polynucleotide extraction device to the amplification device.

In another embodiment, the apparatus further comprises a fraction collector device.

In a preferred embodiment, the fraction collector device is connected to the analysis device by a Fourth connecting means which permits the collection of a quantified product.

In another embodiment, the apparatus further comprises a sequence identifier which identifies the sequence of a quantified product, where the sequence identifier is connected to the analysis device by a fifth connecting means which permits a quantified product to transfer from the analysis device to the sequence identifier.

In another embodiment, the apparatus further comprises a sequence identifier which identifies the sequence of a quantified product, where the sequence identifier is connected to the fraction collector device by a fifth connecting means which permits a collected product to transfer from the fraction collector device to the sequence identifier.

The invention provides an apparatus for expression profiling comprising an amplification device which amplifies a polynucleotide in a reaction mixture to generate an amplified product; an analysis device connected to the amplification device by a first connecting means which permits an aliquot of the reaction mixture to transfer from the amplification device to the analysis device which detects and quantifies the amplified product; and a fraction collector device which permits the collection of a quantified product.

In a preferred embodiment, the fraction collector device is connected to the analysis device by a fourth connecting means which permits the collection of a quantified product.

In one embodiment, the apparatus further comprises a polynucleotide extraction device connected to the amplification device by a second connecting means which permits an extracted polynucleotide sample to transfer from the polynucleotide extraction device to the amplification device.

In another embodiment, the apparatus further comprises a sequence identifier which identifies the sequence of a quantified product, where the sequence identifier is connected to the fraction collector by a fifth connecting means which permits a collected product to transfer from the fraction collector device to the sequence identifier.

The invention provides an apparatus for expression profiling comprising an amplification device which amplifies a polynucleotide in a reaction mixture to generate an amplified product; an analysis device connected to the amplification device by a first connecting means which permits an aliquot of the reaction mixture to transfer from the amplification device to the analysis device which detects and quantifies the amplified product; and a sequence identifier which identifies the sequence of a quantified product, where the sequence identifier is connected to the analysis device by a fifth connecting means which permits a quantified product to transfer from the analysis device to the sequence identifier.

In one embodiment, the apparatus further comprises a polynucleotide extraction device connected to the amplification device by a second connecting means which permits an extracted polynucleotide sample to transfer from the polynucleotide extraction device to the amplification device.

In another embodiment, the apparatus further comprises a fraction collector device.

In a preferred embodiment, the fraction collector device is connected to the analysis device by a fourth connecting means which permits the collection of a quantified product, and where the fraction collector device is also connected to the sequence identifier by another fifth connecting means which permits a collected product to transfer from the fraction collector to the sequence identifier.

In one embodiment, the amplification device and the analysis device also permit sequence identification of a polynucleotide.

The invention further provides an apparatus for expression profiling, comprising: an amplification device which amplifies a polynucleotide in a reaction mixture to generate an amplified product; and a capillary electrophoresis device which detects and quantifies the amplified product, where a capillary of the capillary electrophoresis device is immersed in the reaction mixture to transfer an aliquot of the reaction mixture from the amplification device to the capillary electrophoresis device.

In one embodiment, the apparatus further comprises a polynucleotide extraction device connected to the amplification device by a second connecting means which permits an extracted polynucleotide sample to transfer from the polynucleotide extraction device to the amplification device.

In another embodiment, the apparatus further comprises a fraction collector device.

In a preferred embodiment, the fraction collector device is connected to the capillary electrophoresis device by a fourth connecting means which permits the collection of a quantified product.

In another embodiment, the apparatus further comprises a sequence identifier which identifies the sequence of a quantified product, where the sequence identifier is connected to the capillary electrophoresis device by a fifth connecting means which permits a quantified product to transfer from the capillary electrophoresis device to the sequence identifier.

In another embodiment, the apparatus further comprises a sequence identifier which identifies the sequence of a quantified product, where the sequence identifier is connected to the fraction collector device by a fifth connecting means which permits a collected product to transfer from the fraction collector device to the sequence identifier.

In another embodiment, the amplification device and the capillary electrophoresis device permit sequence identification of a polynucleotide.

In the apparatus of the present invention, the amplification device is preferably a polymerase chain reaction (PCR) amplification device.

Also preferably, the first connecting means permits an aliquot of the reaction mixture to transfer from the amplification device to the analysis device at the end of each PCR cycle.

Preferably, the reaction mixture comprises one or more PCR amplification primers which are chemically linked to an inner wall of a reaction tube or a well of a microtiter plate.

In the apparatus of the present invention, the amplification device preferably also permits reverse transcription to generate cDNAs.

Preferably, one or more primers used for reverse transcription are chemically linked to an inner wall of a reaction tube or a well of a microtiter plate.

Preferably, the apparatus permits the detection and quantification of a signal generated by one or more fluorescent labels.

In some embodiments of the invention, the first, second, fourth, or fifth connecting means is a robotic arm.

In other embodiments of the invention, the first, second, fourth, or fifth connecting means is a tube or a channel.

In some embodiments of the invention, the first, second, fourth, and fifth connecting means are a single connecting means, e.g., a robotic arm, which transfers samples from one device to another.

In one embodiment, an electric current is applied to the first, second, fourth, or fifth connecting means to permit transfer.

In the apparatus of the present invention, the analysis device is preferably a capillary electrophoresis device.

Preferably, the polynucleotide extraction device in the apparatus permits isolating total RNAs or mRNAs from one or more biological materials.

The present invention will find use in wide applications such as biological and biomedical research; identification of therapeutic agents and diagnostic markers; characterization of cells and organisms that underwent genetic modifications; identification of unknown illness; and characterization of DNA and identification of biological samples. Non-limiting examples of such applications include quantitative PCR, real-time PCR, DNA sequencing, transcription profiling and genotyping.

### BRIEF DESCRIPTION OF DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a schematic view of an apparatus for expression profiling according to one embodiment of the invention. The apparatus 10 consists of an amplification device 64 and an analysis device 68 connected to the amplification device 64 by a first connecting means 66.

FIG. 2 is a schematic view of an apparatus for expression profiling according to one embodiment of the invention. The apparatus 10 consists of a polynucleotide extraction device 20, an amplification device 64 and an analysis device 68. A first connecting means 66 connects the amplification device 64 with the analysis device 68, while a second connecting means 40 connects the polynucleotide extraction device 20 with the amplification device 64.

FIG. 3 is a schematic view of an apparatus for expression profiling according to one embodiment of the invention. The apparatus 10 consists of an amplification device 64, an analysis device 68 and a data generation device 120. A first connecting means 66 connects the amplification device 64 with the analysis device 68, a second connecting means 40 connects the polynucleotide extraction device 20 with the amplification device 64, and a third connecting means 80 connects the analysis device 68 with the data generation device 120.

FIG. 4 is a schematic view of an apparatus for expression profiling according to one embodiment of the invention. The apparatus 10 consists of an amplification device 64 and an analysis device 68. The amplification device 64 permits reverse transcription of the polynucleotide prior to the amplification reaction. A first connecting means 66 connects the amplification device 64 with the analysis device 68.

FIG. 5 is a schematic view of an apparatus for expression profiling according to one embodiment of the invention. The apparatus 10 consists of an amplification device 64 and an analysis device 68. The analysis device 68 permits data generation. A first connecting means 66 connects the amplification device 64 with the analysis device 68.

FIG. 6 is a schematic view of an apparatus for expression profiling according to one embodiment of the invention. The apparatus 10 consists of an amplification device 64 and an analysis device 68 which are located in the same housing 60. A first connecting means 66 within the housing 60 connects the amplification device 64 with the analysis device 68.

FIG. 7 is a schematic view of an apparatus for expression profiling according to one embodiment of the invention. The apparatus 10 consists of a polynucleotide extraction device 20, an amplification device 64, an analysis device 68 and a data generation device 120. A first connecting means 66 connects the amplification device 64 with the analysis device 68, a second connecting means 40 connects the polynucleotide extraction device 20 with the amplification device 64, and a third connecting means 80 connects the analysis device 68 with the data generation device 120.

FIG. 8 is a schematic view of an apparatus for expression profiling according to one embodiment of the invention. The apparatus 10 consists of an amplification device 64, an analysis device 68 and a fraction collector device 160. A first connecting means 66 connects the amplification device 64 with the analysis device 68, and a fourth connecting means 140 connects the analysis device 68 with the fraction collector device 160.

FIG. 9 is a schematic view of an apparatus for expression profiling according to one embodiment of the invention. The apparatus 10 consists of an amplification device 64, an analysis device 68 and a sequence identifier 200. A first connecting means 66 connects the amplification device 64 with the analysis device 68, and a fifth connecting means 180 connects the amplification device 64 with the sequence identifier 200.

FIG. 10 is a schematic view of an apparatus for expression profiling according to one embodiment of the invention. The apparatus 10 consists of an amplification device 64, an analysis device 68, a fraction detector device, and a sequence identifier 200. A first connecting means 66 connects the amplification device 64 with the analysis device 68, a fourth connecting means 140 connects the analysis device 68 with the fraction collector device 160, and a fifth connecting means 180 connects the fraction collector device 160 with the sequence identifier 200.

FIG. 11 is a schematic view of an apparatus for expression profiling according to one embodiment of the invention. The apparatus 10 consists of an amplification device 64 and an analysis device 68, where the analysis device 68 also serves as a sequence identifier 200. A first connecting means 66 connects the amplification device 64 with the analysis device 68.

FIG. 12 is a schematic view of an apparatus for expression profiling according to one embodiment of the invention. The apparatus 10 consists of an amplification device 64, an analysis device 68, a sequence identifier 200, and a data generation device 120. A first connecting means 66 connects the amplification device 64 with the analysis device 68, a fifth connecting means 180 connects the amplification device 64 with the sequence identifier 200, and a third connecting means 80 connects the sequence identifier 200 to the data generating device.

FIG. 13 is a schematic view of an expression profiling process using the apparatus according to some embodiments of the invention.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope of the principles of the present invention.

### DETAILED DESCRIPTION OF THE INVENTION

The following terms and definitions are used herein:

“Sample” as used herein refers to a biological material which is isolated from its natural environment and contains a polynucleotide. A “sample” according to the invention may consist of purified or isolated polynucleotide, or it may comprise a biological sample such as a tissue sample, a biological fluid sample, or a cell sample comprising a polynucleotide. A biological fluid includes, but is not limited to, blood, plasma, sputum, urine, cerebrospinal fluid, lavages, and leukophoresis samples. A sample of the present invention may be any plant, animal, bacterial or viral material containing a polynucleotide, or any material derived therefrom.

“Prepared sample” as used herein refers to a preparation derived from a sample for the purpose of isolating or synthesizing a polynucleotide, i.e., a DNA (e.g., genomic DNA or cDNA) or a RNA (e.g., total RNA or mRNA).

“Aliquot” as used herein refers to a sample volume taken from the entire prepared sample or a reaction mixture. An aliquot is less than the total volume of the sample or reaction mixture, and is preferably 1 μl to 5 μl in volume. In one embodiment of the invention, for each aliquot removed, an equal volume of reaction buffer containing reagents necessary for the reaction (e.g., buffers, salts, nucleotides, and polymerase enzymes) is introduced.

“Connecting means” as used herein refers to a means which connects two devices and permit a fluid and/or a signal to transfer from one device to another device.

“Robotic arm”, as used herein, means a device, preferably controlled by a microprocessor, that physically transfers samples, tubes, or plates containing samples from one location to another. Each location can be a unit in a modular apparatus useful according to the invention. An example of a robotic arm useful according to the invention is the Mitsubishi RV-E2 Robotic Arm. Software for the control of robotic arms is generally available from the manufacturer of the arm.

“Reaction chamber” as used herein refers to a fluid chamber for locating reactants undergoing or about to undergo a reaction (e.g., an amplification reaction or an extraction process). A “reaction chamber” may be comprised of any suitable material, i.e., a material that exhibits minimal non-specific adsorptivity or is treated to exhibit minimal non-specific adsorptivity, for example, including, but not limited to, glass, plastic, nylon, ceramic, or combinations thereof. A “reaction chamber” may be connected to at least one connecting means for transferring material in and out of the reaction chamber.

The term “expression” as used herein refers to the production of a protein or nucleotide sequence in a cell or in a cell-free system, and includes transcription into a RNA product, post-transcriptional modification and/or translation into a protein product or polypeptide from a DNA encoding that product, as well as possible post-translational modifications.

“Expression profiling” as used herein refers to the detection of differences in the expression profile between a plurality of samples.

“Difference in the expression profile” as used herein refers to the quantitative (i.e., abundance) and qualitative difference in expression of a gene. There is a “difference in the expression profile” if a gene expression is detectable in one sample, but not in another sample, by known methods for polynucleotide detection (e.g., electrophoresis). Alternatively, a “difference in the expression profile” exists if the quantitative difference of a gene expression (i.e., increase or decrease) between two samples is about 20%, about 30%, about 50%, about 70%, about 90% to about 100% (about two-fold) or more, up to and including about 1.2 fold, 2.5 fold, 5-fold, 10-fold, 20-fold, 50-fold or more. A gene with a difference in the expression profile between two samples is a gene which is differentially expressed in the two samples.

As used herein, “plurality” refers to two or more. Plurality, according to the invention, can be 3 or more, 100 or more, or 1000 or more, for example, up to the number of cDNAs corresponding to all mRNAs in a sample.

“Amplified product” as used herein refers to polynucleotides which are copies of a portion of a particular polynucleotide sequence and/or its complementary sequence, which correspond in nucleotide sequence to the template polynucleotide sequence and its complementary sequence. An “amplified product,” according to the present invention, may be DNA or RNA, and it may be double-stranded or single-stranded.

“Synthesis” and “amplification” as used herein are used interchangeably to refer to a reaction for generating a copy of a particular polynucleotide sequence or increasing in copy number or amount of a particular polynucleotide sequence. It may be accomplished, without limitation, by the in vitro methods of polymerase chain reaction (PCR), ligase chain reaction (LCR), polynucleotide-specific based amplification (NSBA), or any other method known in the art. For example, a polynucleotide amplification may be a process using a polymerase and a pair of oligonucleotide primers for producing any particular polynucleotide sequence, i.e., the target polynucleotide sequence or target polynucleotide, in an amount which is greater than that initially present.

The term “fraction collection”, as used herein, refers to a device intended for collecting liquid samples originating from a slow flowing source, such as a chromatography column or an electrophoresis device, where the composition of the liquid varies over time. Generally, fraction collectors will include a support surface capable of holding a plurality of separate collection tubes and a dispensing head capable of selectively directing the liquid sample to individual collection tubes. In this way, discrete liquid fractions of the sample may be collected in separate tubes for later analysis or use. In capillary electrophoresis, fraction collection may be performed by immersing the end of a capillary and the electrodes to the collection tube containing liquid and applying current to permit a polynucleotide to be eluted into the collection tube.

The term “sequence identifier”, as used herein, refers to a device which can identify the nucleotide identity of a polynucleotide, i.e., DNA sequencing.

“Label” or “detectable label” as used herein refers to any atom or molecule which can be used to provide a detectable (preferably quantifiable) signal, and which can be operatively linked to a polynucleotide. Labels may provide signals detectable by fluorescence, radioactivity, colorimetry, gravimetry, X-ray diffraction or absorption, magnetism, enzymatic activity, mass spectrometry, binding affinity, hybridization radiofrequency, nanocrystals and the like. A primer of the present invention may be labeled so that the amplification reaction product may be “detected” by “detecting” the detectable label. “Qualitative or quantitative” detection refers to visual or automated assessments based upon the magnitude (strength) or number of signals generated by the label.

“Isolated” or “purified” as used herein in reference to a polynucleotide means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95%, up to 99-100% pure) of non-nucleotide or polynucleotide material naturally associated with it, and thus is distinguished from isolated chromosomes.

“cDNA” as used herein refers to complementary or copy polynucleotide produced from a RNA template by the action of RNA-dependent DNA polymerase (e.g., reverse transcriptase). A “cDNA clone” refers to a duplex DNA sequence complementary to an RNA molecule of interest, carried in a cloning vector.

“Genomic DNA” as used herein refers to chromosomal DNA, as opposed to complementary DNA copied from a RNA transcript. “Genomic DNA”, as used herein, may be all of the DNA present in a single cell, or may be a portion of the DNA in a single cell.

The present invention relates to an automated apparatus for gene expression profiling. The apparatus is capable of providing high throughput expression analysis on a plurality of samples, as well as a single sample. A single automated device thus includes in a single system the functions that are traditionally performed by a technician employing pipettors, incubators, polynucleotide amplification device, analysis device (e.g., gel electrophoresis system), and data acquisition systems. The apparatus of the present invention permits the detection, analysis, quantification, and/or visualization of the amplified products.

The practice of the present invention employs, unless otherwise indicated, conventional techniques of molecular biology, microbiology and recombinant DNA techniques, which are known to those skilled in the art and explained in the literature. See, e.g., Sambrook, Fritsch & Maniatis, 1989, Molecular Cloning: A Laboratory Manual, Second Edition; Oligonucleotide Synthesis (M. J. Gait, ed., 1984); Polynucleotide Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995). The practice of the present invention may also involve techniques and compositions as disclosed in U.S. Pat. Nos. 5,965,409; 5,665,547; 5,262,311; 5,599,672; 5,580,726; 6,045,998; 5,994,076; 5,962,211; 6,217,731; 6,001,230; 5,963,456; 5,246,577; 5,126,025; 5,364,521; and 4,985,129. All patents, patent applications, and publications mentioned herein, both supra and infra, are hereby incorporated by reference.

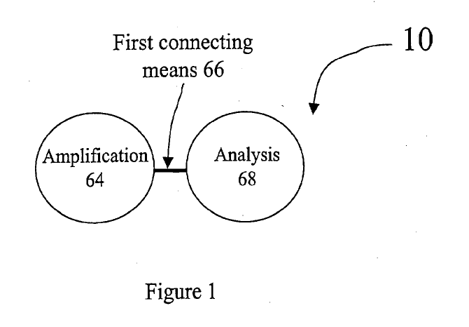

An apparatus for gene expression profiling of the present invention is illustrated generally at 10 in FIG. 1. The apparatus 10 consists of an amplification device 64 and an analysis device 68 connected to the amplification device 64 by a first connecting means 66. A polynucleotide extracted from a sample of interest is amplified in the amplification device 64. An aliquot of the amplified polynucleotide product is then transferred to the analysis device 68 by the first connecting means 66. The analysis device 68 performs the detection and quantification of the amplified product.

In one embodiment, the apparatus permits polymerase chain reaction (PCR) amplification of the polynucleotide, and the amplified product is analyzed by electrophoresis. Preferably, capillary electrophoresis is employed to analyze the amplified products.

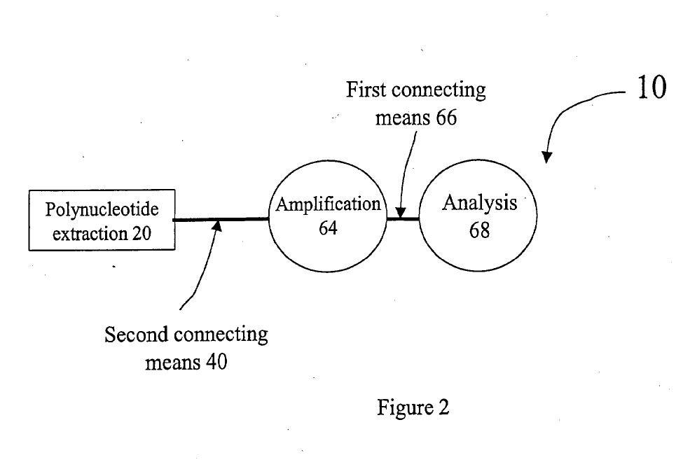

As shown in FIG. 2, the apparatus for expression profiling of the present invention further permits the preparation of DNA templates for the amplification reaction. The apparatus 10 includes a polynucleotide extraction device 20, an amplification device 64 and an analysis device 68. A first connecting means 66 connects the amplification device 64 with the analysis device 68 and a second connecting means 40 connects the polynucleotide extraction device 20 and the amplification device 64. A biological sample is introduced into the polynucleotide extraction device 20 and polynucleotides are extracted from the biological material. The extracted polynucleotides are then transferred to the amplification device 64 through the second connecting means 40 so that the polynucleotides are amplified in the amplification device 64. An aliquot of the amplified polynucleotide products are then transferred to the analysis device 68 by the first connecting means 66. The analysis device 68 performs the detection and quantification of the amplified products.

In a preferred embodiment, the polynucleotide extraction device extracts RNAs from a biological material. In a more preferred embodiment, mRNAs are extracted from a biological material in the polynucleotide extraction device 20.

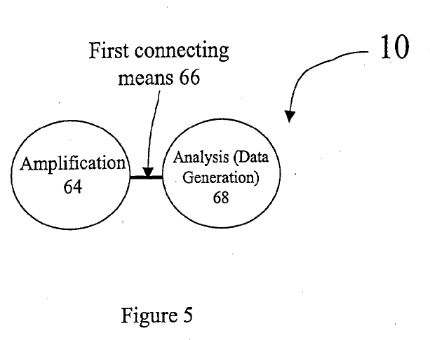

The analysis device 68 of the apparatus may be capable of generating the desired expression profiling data as generally illustrated in FIG. 5. The apparatus 10 consists of an amplification device 64 and an analysis device 68 connected to the amplification device 64 by a first connecting means 66. A polynucleotide extracted from a sample of interest is amplified in the amplification device 64. An aliquot of the amplified polynucleotide product is then transferred to the analysis device 68 by the first connecting means 66. The analysis device 68 performs the detection and quantification of the amplified product, and generates the expression profiling data.

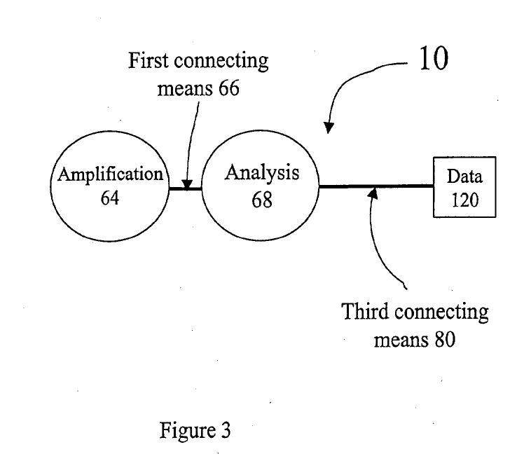

Alternatively, the apparatus for expression profiling of the present invention may further include a separate data generation device as illustrated in FIG. 3. The apparatus 10 consists of an amplification device 64, an analysis device 68 and a data generation device 120. A first connecting means 66 connects the amplification device 64 with the analysis device 68 and a third connecting means 80 connects the analysis device 68 to the data generation device 20.

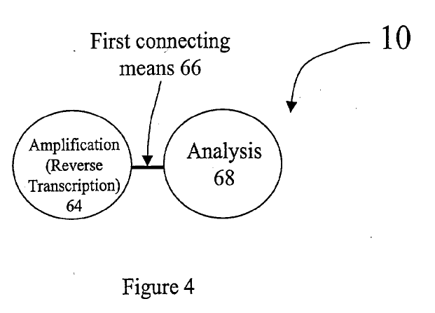

As shown in FIG. 4, the amplification device of the apparatus for expression profiling permits the generation of cDNAs by reverse transcription. The apparatus 10 consists of an amplification device 64 and an analysis device 68. A first connecting means 66 connects the amplification device 64 with the analysis device 68. Extracted RNAs (e.g., total RNAs or mRNAs) are introduced into the amplification device 64 and cDNAs are synthesized from the RNAs within the amplification device 64. The synthesized cDNAs are then amplified in the amplification device 64. An aliquot of the amplified polynucleotide products are then transferred to the analysis device 68 by the first connecting means 66. The analysis device 68 performs the detection and quantification of the amplified products.

### Polynucleotide Extraction Device 20

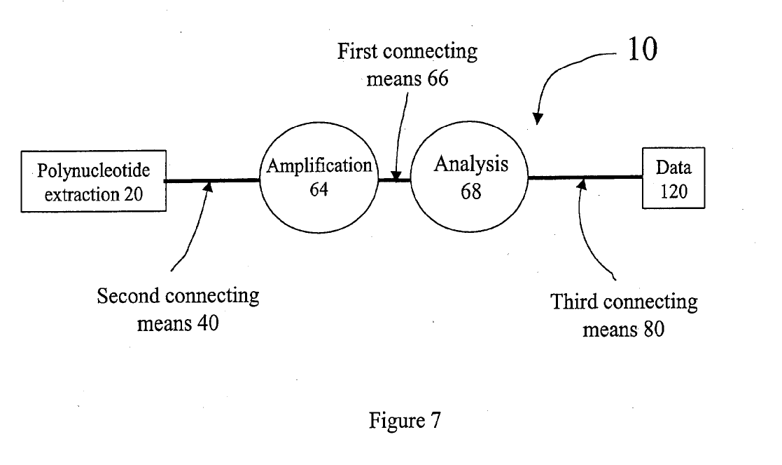

As shown in FIG. 2 and FIG. 7, the polynucleotide extraction device 20 according to the present invention is capable of permitting the direct extraction of polynucleotides (i.e., DNA or RNA) from a biological sample (e.g., a cell sample or a tissue sample).

Preferably, the polynucleotide extraction device 20 is designed to provide the extracted polynucleotide to be used as templates for a reverse transcription reaction and/or a PCR amplification reaction in the amplification device 64. In one embodiment, the polynucleotide extraction device 20 provides the prepared polynucleotide in quality and volumes that correspond to the requirements of existing or future systems for the amplification of polynucleotides. Commercially available amplification systems include, but are not limited to, GeneAmp PCR System 9700 by Applied Biosystems (Forster City, Calif.); iCycler Thermal Cycler by Hercules, Calif.; Eppendorf Mastercycler Gradient by Eppendorf; Smart Cycler TD System by Cepheid (Sunnyvale, Calif.); LightCycler by Roche (Indianapolis, Ind.); AMPLICOR™ automated PCR system (Roche, Indianapolis, Tenn.), and succeeding generations of such instruments. The extraction device can be designed to provide any suitable output volume of fluid that contains the extracted polynucleotide, such as, for example, from about 100 ml to about 750 μl, preferably from about 500 ml to about 500 μl, more preferably from about 1 μl to about 250 μl, more preferably yet from about 1 μl to about 100 μl.

In one embodiment, the polynucleotide extraction device 20 permits the isolation of mRNA from a biological material. In another embodiment, the polynucleotide extraction device 20 permits the isolation of mRNA from a plurality of biological materials.

The technology and reagents for extracting polynucleotides are known in the art, for example, as described in Basic Methods in Molecular Biology, (1986, Davis et al., Elsevier, N.Y.); and Current Protocols in Molecular Biology (1997, Ausubel et al., John Weley & Sons, Inc.).

A variety of polynucleotide extraction apparatuses using the above-described polynucleotide extraction technology can be used in conjunction with the present invention. For example, Japanese Patent Publication No. 125972/1991 describes a polynucleotide extraction apparatus designed to prevent viral infection and improve the efficiency of extraction which comprises a multiarticulated industrial robot and peripheral units necessary for DNA extraction and purification. Japanese Patent Publication No. 131076/1992 discloses an extraction apparatus designed to improve the efficiency of extraction of polynucleotides from a small amount of blood or other biological material through a compact arrangement of means for transfer of the polynucleotide extraction vessel to a centrifuge. Japanese Patent Publication No. 47278/1997 discloses an extraction apparatus employing a filter system equipped with a vacuum pump in lieu of a centrifuge. In order that a fully automatic extraction device may be implemented, a centrifuge or a vacuum pump and the associated hardware may be built into the device.

in one embodiment, the polynucleotide extraction device 20 is a polynucleotide extraction apparatus. The polynucleotide extraction apparatus of the present invention may comprise (1) a group of extraction vessels each comprising a reactor tube in which a biological material, a reagent solution, and a magnetic carrier are admixed and reacted, a drain cup for pooling an unwanted component solution, and a polynucleotide recovery tube all as secured to a support, (2) a distribution means for introducing a solution into each of the extraction vessels, (3) a stirring means for mixing the solution and magnetic carrier in the reactor tube, (4) a holding means for holding the magnetic carrier stationary within the vessel, (5) a discharging means for discharging the solution from the reactor tube while the magnetic carrier is held stationary, (6) a heating means for heating the solution and magnetic carrier in the reactor tube, and (7) a transfer means for serially transferring the vessels to the given positions. Such a device is described in U.S. Pat. No. 6,281,008 hereby incorporated by reference in its entirety.

In another embodiment, the polynucleotide extraction device 20 is an automated polynucleotide isolation device. The device comprises a removable cassette, where the cassette comprises a separable sample transfer/storage strip. The cassette can be sealed or open, preferably it is sealed. The preferred cassette also has a movable input transfer bar, and is encased in a caddy. The device may further comprise a hollow body having a top side, an exterior, an interior, at least one slot for the placement of the cassette, and at least one well for the placement of a sample container. Additionally, the cassette includes a means for moving the cassette from or into the caddy, as well as a means for activating the input transfer sample bar. The preferred device also comprises an air nozzle in communication with means for accessing, storing, or generating pressurized air, and a means for sealing sample input channels of the cassette. Furthermore, the device includes valve actuators located in the interior for opening and closing valves in the cassette, and one or more pump actuators for moving fluid in or out of fluid chambers in the cassette. The device also preferably includes a magnet, a power supply, a user interface, and a bar-code reading means. Preferably, the device also comprises a sensor means in the slot or well, which signals that the slot or well is occupied when a cassette or sample container has been respectively inserted therein. Such a device is described in U.S. Pat. No. 6,281,008 hereby incorporated by reference in its entirety.

In another embodiment, the polynucleotide extraction device 20 further comprises a memory means. In another embodiment, the polynucleotide extraction device 20 further comprises a separating means for separating the strip from the remainder of the cassette. The separating means is preferably a knife having a heating means in communication thereto, the use of which seals both the strip and the remainder of the cassette. The preferred device has more than one well; more preferred, the device has about 24 wells or 48 wells or 96 wells or 386 wells. The device preferably includes the cassette that further comprises: (1) one or more sample entry ports located on the input transfer sample bar that are serially and respectively in communication with the same number of wells of the device, where the ports are also in communication with input sample storage reservoirs of the cassette; (2) one or more reaction flow-ways that are serially and respectively in communication via fluid exchange channels with the same number of sample input storage reservoirs; (3) fluid chambers in communication with the fluid exchange channels, wherein fluid chambers are supply chambers for reagents, reservoirs for samples, or reaction chambers; (4) valves for controlling the flow of fluids in the fluid exchange channels; and (5) a sample transfer/storage strip having at least one of the fluid chambers that is in communication with a reaction flow-way.

The polynucleotide extraction device 20 is designed for the preparation of polynucleotide from any biological sample. A biological sample used in the context of the present invention is any material that contains polynucleotide, i.e., RNA or DNA. Such a sample can be an entire organism, such as an insect, or a number of organisms, such as in the analysis of bacteria or yeast; or the sample can be a portion of an organism, such as a tissue, body fluid, or excretion. Suitable tissues from which a polynucleotide composition can be obtained includes, but is not limited to, skin, bone, liver, brain, leaf, root, and the like; i.e., any tissue of a living or deceased organism. The tissue can be substantially uncontaminated with other tissues of the source organism, or it can be so contaminated, or even contaminated with tissues derived from different organisms. Preferably, the source of the organism or organisms from which a particular biological sample is taken is known prior to subjecting it to the method of the present invention; however, such knowledge is not always available, as in the instance of forensic samples.

Biological samples can also be clinical samples or specimens. For example, evidence of a disease or condition caused by an exogenous source can be examined by testing the polynucleotide taken from a sample of a certain clinical specimen, such as urine, fecal matter, spinal fluid, sputum, blood or blood component, or any other suitable specimen, for the presence of a particular pathogen, for example, as evidenced by the identification in the preparation of characteristic polynucleotide sequences contained within such a pathogen. The existence or propensity for certain inborn genetic diseases or conditions in an individual can also be tested. Such genetic diseases include, but are not limited to, Huntington's disease, Tay Sach's disease, and others, by testing for polynucleotide sequences characteristic of such genetic diseases or propensities in the polynucleotide isolated from suitable clinical samples, such as any cellular matter of the tested individual, with the caveat that cells having rearranged or detectably less DNA with respect to that of germ line stem cells, such as red blood and antibody-forming cells, alone may not be sufficient for such a test.

The polynucleotide extracted, i.e., isolated, by the polynucleotide extraction device therefor is any suitable polynucleotide, where the suitability is determined by the type of test desired. For example, for testing for the presence of a certain pathogen in an individual, preferably one would test for an identifying polynucleotide sequence or sequences found in a DNA composition taken from a clinical sample where the known biology of the pathogen and host would suggest that the pathogen would be found if the tested individual were so infected. Alternatively, for testing whether a particular gene is being expressed in an individual, one can test for such expression by seeking evidence of an identifying polynucleotide sequence or sequences in an RNA composition taken from a tissue in which the underlying biology/pathology indicates that the expression should or should not be found, as appropriate to the condition or disease being tested. Depending on the gene whose expression is being monitored, the RNA composition can be further refined to include predominantly polyadenylated or non-polyadenylated RNA species using methods known in the art. Alternatively, or additionally, size classes of RNA species can be selected for in the context of the present invention as well.

Biological samples can be freshly taken from an individual or isolated from nature, or such samples can be stored using suitable conditions, such as on ice. For example, a sample of blood can be collected from an individual using standard means, such as a hypodermic needle placed into an individual's vein and connected to a standard evacuated tube, for example, to draw the blood from the individual into the tube. The blood can be used directly or stored on ice, preferably in the presence of an anti-coagulant, such as heparin, citrate, or EDTA. For longer storage, the samples are preferably frozen, freeze-dried, or applied to a suitable substrate and dried thereon for storage of, for example, DNA. Such a suitable substrate includes any absorbent paper, such as a Whatman filter paper, or a treated membrane material that releasably binds DNA, A preferred membrane is included in a commercial product named IsoCode™ Stix (Schleicher & Schuell, Inc., Keene, N. H.), which, in addition to reversibly binding DNA, also irreversibly binds hemoglobin (an inhibitor of certain polynucleotide amplification methods). The substrate-bound polynucleotide can then be extracted from the substrate and purified in the same fashion as a fresh sample, in accordance with the present invention.

Preferably, the polynucleotide extraction device 20 permits nucleic acid extraction from one or more biological samples. In one embodiment, this is achieved by such device comprising a removable cassette that is insertable into a slot in the device. Preferably, the device includes slots for four different cassettes (e.g., each cassette for a sample) that can be run concurrently, serially, or in a staggered fashion.

The sample preparation device may also serve as a reservoir of the amplification reaction mixture so that an amount equivalent to the aliquot is replenished into the reaction mixture after each transfer of amplified products to the analysis device.

### Amplification Device 64

As shown in FIG. 1, the amplification device 64 according to the present invention may be any device capable of amplifying a polynucleotide, preferably through a polynucleotide chain reaction (PCR) reaction. Typically, PCR reaction is performed by a thermal cycler. Useful thermal eyelets include, but are not limited to, GeneAmp PCR System 9700 by Applied Biosystems (Forster City, Calif.); iCycler Thermal Cycler by Bio-Rad (Hercules, Calif.); Eppendorf Mastercycler Gradient by Eppendorf; Smart Cycler TD System by Cepheid (Sunnyvale, Calif.); LightCycler by Roche (Indianapolis, Ind.); AMPLICOR™ automated PCR system (Roche, Indianapolis, Ind.). PCR devices useful according to the present invention include, but are not limited to, those described in U.S. Pat. Nos. 5,475,610; 5,602,756; 5,720,923; 5,779,977; 5,827,480; 6,033,880; and 6,326,147; 6,1716,785, all of which are incorporated hereby by reference in their entireties.

The purpose of a polymerase chain reaction is to manufacture a large amount of DNA which is identical to an initially supplied small volume of “template” DNA. The reaction involves copying the strands of the DNA and then using the copies to generate other copies in subsequent cycles. Under ideal conditions, each cycle will double the amount of DNA present thereby resulting in a geometric progression in the volume of copies of the “target” or “template” DNA strands present in the reaction mixture.

For example, a typical PCR temperature cycle requires that the reaction mixture be held accurately at each incubation temperature for a prescribed time and that the identical cycle or a similar cycle be repeated many times. A typical PCR program starts at a sample temperature of about 94° C. held for about 30 seconds to denature the reaction mixture. Then, the temperature of the reaction mixture is lowered to about 30° C. to about 60° C. and held for one minute to permit primer hybridization. Next, the temperature of the reaction mixture is raised to a temperature in the range from about 50° C. to about 72° C. where it is held for about two minutes to promote the synthesis of extension products. This completes one cycle. The next PCR cycle then starts by raising the temperature of the reaction mixture to about 94° C. again for strand separation of the extension products formed in the previous cycle (denaturation). Typically, the cycle is repeated 25 to 30 times. It is understood in the art that the temperatures of a PCR cycle and the number of cycles in a PCR reaction vary according to the objectives of the reaction and the characteristics of the template, e.g., ™. The basic PCR protocols and strategies are known in the art, for example, as described in Basic Methods in Molecular Biology, (1986, Davis et al., Elsevier, N.Y.); and Current Protocols in Molecular Biology (1997, Ausubel et al., John Weley & Sons, Inc.).

In one embodiment, the reaction mixture is stored in a disposable plastic tube which is closed with a cap. A typical sample volume for such tubes is about 50-100 microliters. Typically, such device uses many tubes filled with sample DNA and reaction mixture inserted into holes called sample wells in a metal block. To perform the PCR process, the temperature of the metal block is controlled according to prescribed temperatures and times specified by the user in a PCR protocol file. A computer and associated electronics then controls the temperature of the metal block in accordance with the user supplied data in the PCR protocol file defining the times, temperatures and number of cycles, etc. As the metal block changes temperature, the samples in the various tubes follow with similar changes in temperature.

Generally, it is desirable to generate uniformity of temperature from place to place within the metal block because temperature gradients existing within the metal of the block may cause some samples to have different temperatures than other samples at particular times in the cycle. It is also desirable to minimize delays in transferring heat from the sample block to the sample especially because the delays are not the same for all samples. These factors are considered when designing the PCR device of the present invention.

In one embodiment, the PCR device has a metal block which is large enough to accommodate 96 sample tubes arranged in the format of an industry standard microtiter plate. The microliter plate is a widely used means for handling, processing and analyzing large numbers of small samples in the biochemistry and biotechnology fields. Useful microtiter plates may contain 24 wells, 48 wells, 96 wells, 196 wells, or 384 wells. Typically, a microtiter plate is a tray which is 3⅝ inches wide and 5 inches long and contains 96 identical sample wells in an 8 well by 12 well rectangular array on 9 millimeter centers. Microliter plates are available in a wide variety of materials, shapes and volumes of the sample wells, which are optimized for many different uses. Preferably, the microtiter plates have the overall outside dimensions and the same 8×12 array of wells on 9 millimeter centers. A wide variety of equipment is available for automating the handling, processing and analyzing of samples in this standard microliter plate format. Microtiter plates are commercially available in the art, for example, from MWG biotech Inc. (High Point, N.C.). The microplate may be made by methods known in the art, for example, as described in U.S. Pat. No. 5,602,756, which is hereby incorporated by reference.

Preferably, the tubes used for the microtiter plate are thin walled sample tubes for decreasing the delay between changes in sample temperature of the sample block and corresponding changes in temperature of the reaction mixture. The wall thickness of the section of the sample tube which is in contact with whatever heat exchange is being used should be as thin as possible so long as it is sufficiently strong to withstand the thermal stresses of PCR cycling and the stresses of normal use. Typically, the sample tubes are made of autoclavable polypropylene such as Himont PD701 with a wall thickness of the conical section in the range from 0.009 to 0.012 inches plus or minus 0.001 inches.

In another embodiment, the PCR device employs heating and cooling a sample block which results in sample-to-sample uniformity despite rapid thermal cycling rates, noncontrolled varying ambient temperatures and variations in other operating conditions such as power line voltage and coolant temperatures. A heated cover may be used to prevent condensation and sample volume loss as described below.

In another embodiment, the PCR device prevents the loss of solvent from the reaction mixtures when the samples are being incubated at temperatures near their boiling point. A heated platen covers the tops of the sample tubes and is in contact with an individual cap which provides a gas-tight seal for each sample tube. The heat from the platen heats the upper parts of each sample tube and the cap to a temperature above the condensation point such that no condensation and refluxing occurs within any sample tube. Condensation represents a relatively large heat transfer since an amount of heat equal to the heat of vaporization is given up when water vapor condenses. This could cause large temperature variations from sample to sample if the condensation does not occur uniformly. The heated platen prevents any condensation from occurring in any sample tube thereby minimizing this source of potential temperature errors. The use of the heated platen also reduces reagent consumption.

In a preferred embodiment, the amplification device 64 of the present invention permits the performance of reverse transcription to synthesize cDNAs. Reverse transcription reaction refers to an in vitro enzymatic reaction in which the template-dependent polymerization of a DNA strand complementary to an RNA template occurs. Reverse transcription is performed by the extension of an oligonucleotide primer annealed to the RNA template, and most often uses a viral reverse-transcriptase enzyme, such as AMV (avian myeloblastosis virus) reverse transcriptase or MMLV (Moloney murine leukemia virus) reverse transcriptase. Conditions and methods for reverse transcription are known in the art. Exemplary conditions for reverse transcription include the following: for AMV reverse transcriptase—reaction at about 37° C. in buffer containing 50 mM Tris-HCl, pH 8.3, 75 mM KCl, 3 mM MgCl₂, 10 mM DTT, 0.8 mM dNTPs, 50 units of reverse transcriptase, and 1-5 μg of template RNA; for MMLV reverse transcriptase—reaction at 37° C. in buffer containing 50 mM Tris-HCl, pH 8.3, 30 mM KCl, 8 mM MgCl₂, 10 mM DTT, 0.8 mM dNTPs, 50 units of reverse transcriptase, and 1-5 μg of template RNA.

In another preferred embodiment, the reverse transcription is performed with a 96 well plate, where the cDNAs are synthesized by using one or more oligonucleotide primers chemically linked to the inner wall of the plate wells. Techniques for synthesizing such chemically linked oligonucleotides are disclosed in McGall et al., International application No. PCT/US93/03767; Pease et al., (1994) Proc. Natl. Acad. Sci., 91: 5022-5026; Southern and Maskos, International application PCT/GB89/01114; Maskos and Southern (Supra); Southern et al., (1992) Genomics, 13: 1008-1017; and Maskos and Southern, (1993) Polynucleotides Research, 21: 4663-4669, each of which is hereby incorporated by reference in its entirety.

In some embodiments, the reverse transcription is performed using one or more oligonucleotides chemically attached to the inner wall of wells of the microliter plate. In other embodiments, the amplification reaction is performed using at least one oligonucleotide primer chemically linked to the inner wall of wells of the microtiter plate or reaction tube. As a result, the synthesized cDNAs or amplified polynucleotide products are attached to the inner wall of the microtiter plate for easy separation and purification.

Oligonucleotides may also be synthesized on a single (or a few) solid phase support such as the inner wall of wells of the microtiter plate or a reaction tube to form an array of regions uniformly coated with synthesized oligonucleotides. Techniques for synthesizing such arrays are disclosed in McGall et al., International application PCT/US93/03767; Pease et al., (1994) Proc. Natl. Acad. Sci., 91: 5022-5026; Southern and Maskos, International application PCT/GB89/01114; Maskos and Southern (Supra); Southern et al., (1992) Genomics, 13: 1008-1017; and Maskos and Southern, (1993) Polynucleotides Research, 21: 4663-4669.

In one embodiment, the amplification device generates labeled amplified products. For example, amplified products may be generated by using a labeled primer. A labeled polynucleotide (e.g., an oligonucleotide primer) according to the methods of the invention is labeled at the 5′ end, the 3′ end, or both ends, or internally. The label can be “direct”, e.g., a dye, radioactive label. The label can also be “indirect”, e.g., antibody epitope, biotin, digoxin, alkaline phosphatase (AP), horse radish peroxidase (HRP). For detection of “indirect labels” it is necessary to add additional components such as labeled antibodies, or enzyme substrates to visualize the captured, released, labeled polynucleotide fragment. In a preferred embodiment, an oligonucleotide primer is labeled with a fluorescent label. Suitable fluorescent labels include fluorochromes such as rhodamine and derivatives (such as Texas Red), fluorescein and derivatives (such as 5-bromomethyl fluorescein), Lucifer Yellow, IAEDANS, 7-Me₂N-coumarin-4-acetate, 7-OH-4-CH₃-coumarin-3-acetate, 7-NH₂-4-CH₃-coumarin-3-acetate (AMCA), monobromobimane, pyrene trisulfonates, such as Cascade Blue, and monobromorimethyl-ammoniobimane (see, for example, DeLuca, Immunofluorescence Analysis, in Antibody As a Tool, Marchalonis, et al., eds., John Wiley & Sons, Ltd., (1982), which is hereby incorporated by reference).

### Analysis Device 68—Capillary Electrophoresis Device

Capillary electrophoresis is the preferred method for analyzing the amplified products of the present invention. As shown in FIG. 1, the present invention provides a single apparatus which comprises both the amplification device 64 and the analysis device 68, e.g., a capillary electrophoresis device. Capillary electrophoresis devices are known in the art. Capillary electrophoresis devices useful according to the invention include, but are not limited to, ABI PRISM® 3100 Genetic Analyzer, ABI PRISM® 3700 DNA Analyzer, ABI PRISM® 377 DNA Sequencer, ABI PRISM® 310 Genetic Analyzer by Applied Biosystems (Foster City, Calif.); MegaBACE 1000 Capillary Array Electrophoresis System by Amersham Pharmacia Biotech (Piscataway, N.J.). CEQ™ 8000 Genetic Analytic System by Beckman Coulter (Fullerton, Calif.); Agilent 2100 Bioanalyzer by Caliper Technologies (Mountain View, Calif.); iCE280 System by Convergent Bioscience Ltd. (Toronto, Canada). Capillary electrophoresis repeat devices useful may be on as described in U.S. Pat. Nos. 6,217,731; 6,001,230; 5,963,456; 5,246,577; 5,126,025; 5,364,521; 4,985,129; 5,202,010; 5,045,172; 5,560,711; 6,027,624; 5,228,969; 6,048,444; 5,616,228; 6,093,300; 6,120,667; 6,103,083; 6,132,582; 6,027,627; 5,938,908; and 5,916,428, all of which are hereby incorporated by reference in their entireties.

In capillary electrophoresis, two reservoirs containing the background electrolyte solution are interconnected by a capillary tube which contains the same solution. Each reservoir is equipped with an electrode. The sample to be analyzed is introduced as a short zone into one end of the capillary. For the introduction of a sample the end of the capillary is usually transferred into one reservoir, and the desired amount of the sample solution is injected into the capillary, where-after the capillary end is transferred back into the background solution. By means of electrodes in the reservoirs, an electric field is applied on the capillary, usually ranging from 200 to 1000 V/cm, under the effect of which the electrically charged particles will begin to move in the capillary. The different particles will separate from each other if they have different speeds in the electric field. The particle zones will pass a detector at the other end of the capillary at different times, and their signals are measured.

In one embodiment, the capillary electrophoresis device provides a plurality of capillaries, an electrode/capillary array, multilumen tubing, tubing holders, optical detection region, capillary bundle and high pressure T-fitting. The capillaries have sample ends disposed in the electrode/capillary array and second ends received by the high pressure T-fitting.

Preferably, the electrode/capillary array includes electrodes and the sample ends of capillaries protruding from the bottom side of the capillary electrophoresis device. The electrodes and the sample ends of capillaries are arranged to be dipped into corresponding sample wells in a 96-well or a 384-well microtiter tray; this requires 96 or 384 capillaries in order to fully utilize every well on the microtiter tray.

Also preferably, the capillaries run inside of corresponding multilumen tubes which are firmly fixed in place by the tubing holders. Exposed portions of the capillaries, lined up side-by-side and without the protection of multilumen tubing, then pass through the optical detection region, which includes a camera assembly. The camera assembly captures images of samples traveling inside the exposed capillaries. The exposed second ends of the capillaries are then bundled together and fitted into the high pressure T-fitting.

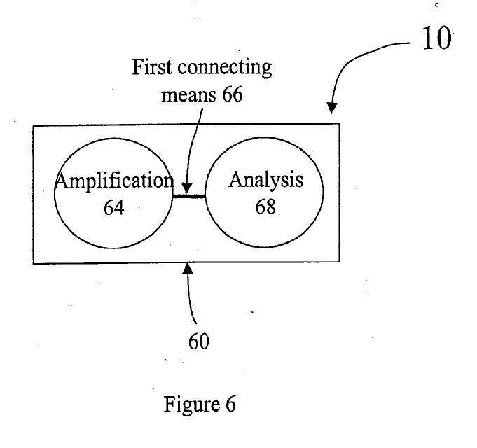

In one embodiment, the amplification device 64 and the analysis device 68 are located in the same housing 60 as shown in FIG. 6. A first connecting means 66 within the housing 60 connects the amplification device 64 with the analysis device 68.

### Data Generation 120

As shown in FIG. 5, the analysis device 68 of the present invention may permit data generation. Alternatively, the data may be generated by a separate data generation device 120 as illustrated in FIG. 3.

Data generation may be achieved by method known in the art, for example, as described in U.S. Pat. Nos. 6,217,731; 6,001,230; 5,963,456; 5,246,577; 5,126,025; 5,364,521; 4,985,129; 5,202,010; 5,045,172; 5,560,711; 6,027,624; 5,228,969; 6,048,444; 5,616,228; 6,093,300; 6,120,667; 6,103,083; 6,132,582; 6,027,627; 5,938,908; 5,900,934; 6,184,990; and 5,916,428, all of which are hereby incorporated by reference in their entireties.

In one embodiment, the data generation device comprises a signal detector, a display monitor and a computer processor coupled to the control circuit and the display monitor. The computer processor includes an input/output (I/O) interface configured to communicate with a control circuit and a first computer memory storing a display program which displays a graphical user interface on the display monitor.

Preferably, the data generation device permits the detection and quantification of fluorescent signals generated by fluorophores. Fluorophores include, but are not limited to, rhodamine and derivatives (such as Texas Red), fluorescein and derivatives (such as 5-bromomethyl fluorescein), Lucifer Yellow, IAEDANS, 7-Me₂N-coumarin-4-acetate, 7-OH-4-CH₃-coumarin-3-acetate, 7-NH₂-4-CH₃-coumarin-3-acetate (AMCA), monobromobimane, pyrene trisulfonates, such as Cascade Blue, and monobromorimethyl-ammoniobimane.

In one embodiment, the device provides a concave reflector positioned at one side of the capillary flow cell as a first high numerical aperture (N.A.) collector, a lens collector positioned at an opposite side of the flow cell as a second high N.A. collector, and an optical fiber positioned at close proximity of the flow cell for delivery of an excitation light to cause a sample contained in the flow cell to emit emission lights. The reflector has a concave surface for reflecting the emission lights, and the collector has a proximal convex surface for collecting the emission lights, and a distal convex surface for collimating the emission lights. This arrangement achieves a larger solid collection angle from both sides of the flow cell and therefore an increased collection efficiency. Two or more optical fibers may be used to deliver excitation lights from different sources. The optical fibers are arranged in a plane orthogonal to the optical axis of the reflector and collector to reduce the interference from the scattered background lights and therefore improve the signal to noise ratio. The collimated emission lights can be detected by, e.g., a photo-multiplier tube detector.

### Fraction Collector 160

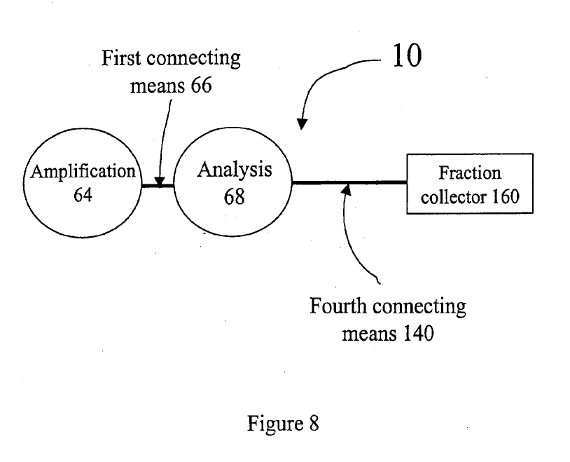

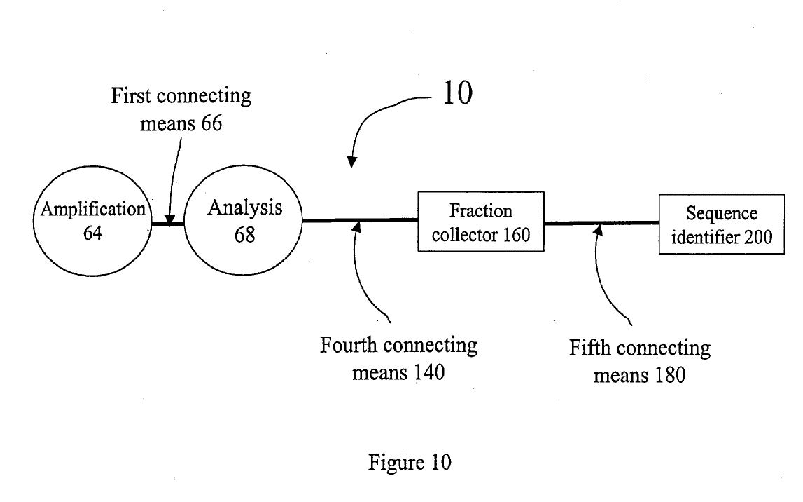

In the present invention, the apparatus may comprise a fraction collector which is connected to the analysis device to collect any desired polynucleotide samples from the analysis device. As shown in FIG. 8, the fraction collector 160 may be connected to the analysis device 68 though a fourth connecting means 140. In addition, as shown in FIG. 10, the fraction collector 160 may also be connected to a sequence identifier 200 by a fifth connecting means 180.

Traditionally, fraction collectors may be broadly categorized into two groups. In the first group, the collection tubes are arranged in a generally rectangular array and the dispensing head is manipulated to selectively feed the individual collection tubes. In the second group, the collection tubes are arranged in a spiral pattern and mounted on a generally circular turntable. The turntable is rotated as the dispensing head is moved radially in order to follow the spiral pattern and track the individual collection tubes. Any of these fraction collectors may be employed in the present invention. Examples of such fraction collectors include, but are not limited to, those disclosed in U.S. Pat. Nos. 4,862,932; 3,004,567; 3,945,412; 4,495,975; 4,171,715, each of which is hereby incorporated by reference in its entirety.

Fraction collectors have been developed to accommodate the needs for high throughput analytical systems and these collectors may also be integrated into the apparatus of the present invention. For example, U.S. Pat. No. 6,309,541 (hereby incorporated by reference in its entirety) discloses an automated fraction collection assembly that retains the microtiter plates in a fixed position and dispenses the sample portions into the selected wells in the microtiter plates. The fraction collection assembly includes a dispensing needle through which the sample portion is dispensed into disposable expansion chambers and then into the microtiter plate. The dispensing needle is mounted on a dispensing head adapted to extend into a disposable expansion chamber into which the sample portion is condensed and then dispensed into the microtiter plate.

Another type of fraction collector useful in the present invention is fraction collectors by electrophoresis, for example, as described in U.S. Pat. Nos. 5,541,420; 5,635,045; 5,439,573; 4,964,961; 4,608,147; 4,049,534; 4,040,940; 3,989,612 (each patent is hereby incorporated by reference in its entirety). In one embodiment, the fraction collector according to the present invention comprises one or more electrophoresis tracks at the specified gap to separate samples by electrophoresis, and the separated components are then eluted from the electrophoresis tracks. One or more capillary sample transferring tubes, which are placed with their ends close to the ends of the electrophoresis tracks at the specified gap, transfer the separated components eluted from each electrophoresis track. Optionally, a connecting means is used to supply the buffer solution to the gap and to carry the separated component to the sample transferring tube by sheath flow of the buffer solution.

Other useful fraction collector devices include, but are not limited to, U.S. Pat. Nos. 6,106,710; 6,004,443; 5,205,154; and 6,355,164, each of which is hereby incorporated by reference in its entirety.

### Sequence Identifier 200

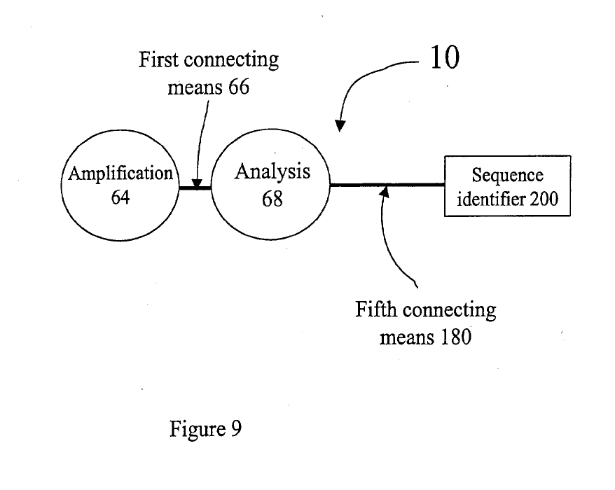

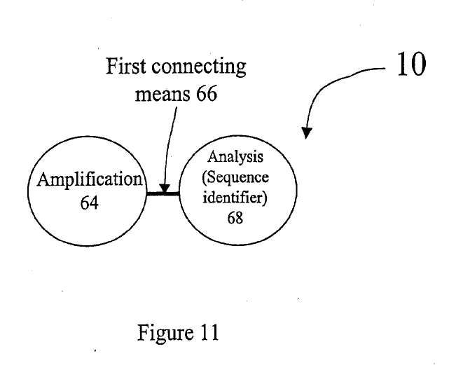

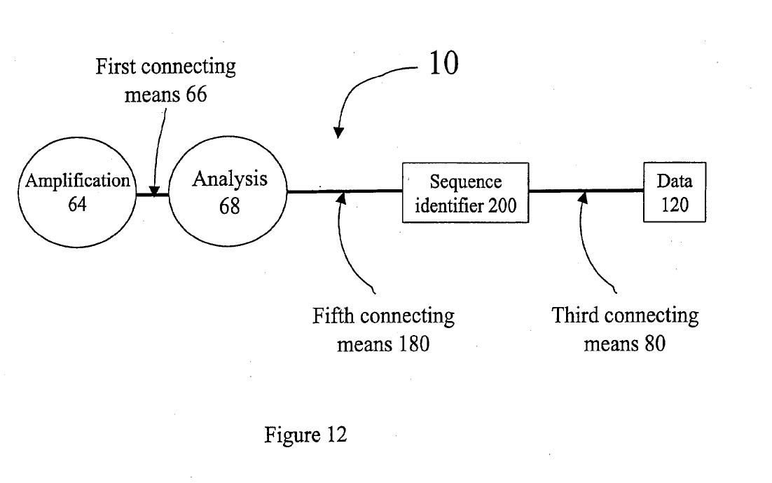

The apparatus of the present invention may further comprise a sequence identifier to provide the sequence of a desired polynucleotide, for example, a polynucleotide of interest identified by the analysis device. As shown in FIG. 10, the sequence identifier 200 may be connected with a fraction collector 160 to identify the sequence of polynucleotide in each fraction collected. In another embodiment as shown in FIGS. 9 and 12, the sequence identifier 200 is connected to the analysis device through a fifth connecting means 180. In another embodiment as shown in FIG. 11, the analysis device 68 itself may serve as the sequence identifier. Preferably, a sample containing a polynucleotide of interest is reloaded onto the analysis device for the identification of its sequence. DNA sequencing is generally carried out by the method of Sanger et al. (Proc. Nat. Acad. Sci. USA 74:5463, 1977) and involves enzymatic synthesis of single strands of DNA from a single stranded DNA template and a primer. A single stranded template is provided along with a primer which hybridizes to the template. The primer is elongated using a DNA polymerase, and each reaction terminated at a specific base (guanine, G, adenine, A, thymine, T, or cytosine, C) via the incorporation of an appropriate chain terminating agent, for example, a dideoxynucleotide. The nucleotide identity of a polynucleotide is then determined according to the chain terminating agent incorporated at each position of the polynucleotide. However, other DNA sequencing devices and methods have also been developed and may be used as the sequence identifier in the present invention.

In a preferred embodiment, there is no separate sequence identifier in the apparatus. The amplification device and the analysis device (e.g., a capillary electrophoresis device) perform the function of sequence identification. Sequencing reagent mixture may be added to the amplification reaction to perform the sequencing reaction and an aliquot of the sequencing reaction is then transferred to the analysis device (e.g., capillary electrophoresis device) for sequence identification. Methods and reagents for sequencing reaction and sequence identification are well known in the art, e.g., in Short Protocols In Molecular Biology, (Ausubel et al., ed., 1995, supra).

Sequence identifiers useful for the present invention may include, but are not limited to, those disclosed in U.S. Pat. Nos. 6,270,961; 6,025,136; 5,955,030; 5,846,727; 5,821,058; 5,608,063; 5,643,798; 5,556,790; 5,453,247; 5,332,666; 5,306,618; 5,288,644; 5,242,796; 5,221,518; and 5,122,345, each of which is hereby incorporated by reference in its entirety.

The identified sequence of the polynucleotide of interest may be used to compare with available sequences in various databases, such as Genbank.

### Connecting Means 40, 66, 80, 140 or 180

A connecting means 40, 66, 80, 140 or 180 of the present invention allows fluid and/or signal communication between two devices as illustrated in FIGS. 1-12. Preferably, a connecting means of the present invention can be moved both horizontally and vertically to permit the transfer of fluids. A connecting means may be a tube or a channel, or a robotic arm. A connecting means may comprise two or more tubes. The two or more tubes may be bounded together. The compartment to which the connecting means attaches, e.g., the reaction chamber of the amplification device, may be closed except for the presence of the connecting means, or may have one or more open sides while still defining a volume useable consistent with the goals and objects of this invention. The samples may be transferred electrokinetically through the connecting means, e.g., by using a voltage controller capable of applying selectable voltage levels, including ground. Such a voltage controller can be implemented using multiple voltage dividers and multiple relays to obtain the selectable voltage levels. The use of electrokinetic transport is a viable approach for sample manipulation and as a pumping mechanism. The present invention also entails the use of electroosmotic flow to mix various fluids in a controlled and reproducible fashion. When an appropriate fluid is placed in a tube made of a correspondingly appropriate material, functional groups at the surface of the tube can ionize. Electroosmosis can be used as a programmable pumping mechanism.

Pumping action can also be achieved using, for instance, peristaltic pumps, mechanisms whereby a roller pushes down on the flexible film of a fluid chamber to reduce the volume of the chamber, plungers that press on the flexible film of a fluid chamber to reduce its volume, and other pumping schemes known to the art. Such mechanisms include micro-electromechanical devices such as reported by Shoji et al., “Fabrication of a Pump for Integrated Chemical Analyzing Systems,” Electronics and Communications in Japan, Part 2, 70, 52-59 (1989) or Esashi et al., “Normally closed microvalve and pump fabricated on a Silicon Wafer,” Sensors and Actuators, 20, 163-169 (1989).

The connecting means 40, 66, 140 or 180 useful for the invention may be a robotic arm. A robotic arm physically transfers samples, tubes, or plates containing samples from one location to another. An automated sampling process can be readily executed as a programmed routine and avoids both human error in sampling (i.e., error in sample size and tracking of sample identity) and the possibility of contamination from the person sampling. Robotic arms capable of withdrawing aliquots from thermal cyclers are available in the art. For example, the Mitsubishi RV-E2 Robotic Arm can be used in conjunction with a SciClone™ Liquid Handler or a Robbins Scientific Hydra 96 pipettor. Preferably, the robotic arm of the invention also include a motorized stage that permits both horizontal and vertical movements for the purpose of transferring samples.

In one embodiment, a first connecting means 66 connects the amplification device 64 with the analysis device 68 so that fluids are transported and subjected to a particular analysis. In a preferred embodiment, the first connecting means 66 permits the automatic loading of a fluid sample to a loading well within the analysis device 68. The volume or “plug” of sample that is disposed within the loading well is then drawn down the analysis channel whereupon it is subjected to the desired analysis. In a preferred embodiment, the analysis device 68 is a capillary electrophoresis device. Accordingly, for such operations, the main or analysis channel generally includes a sieving matrix, buffer or medium disposed therein, to optimize the electrophoretic separation of the constituent elements of the sample. However, it will be appreciated upon reading the instant disclosure that the analysis device 68 may also be a wide variety of non-CE devices, and may be used to perform any of a number of different analytical reactions on a sample.

Preferably, the connecting means 66 for transferring samples permits withdrawing an aliquot from an amplification reaction during the amplification regimen. The connecting means 66 may comprise pipette tips or needles that are either disposed of after a single sample is withdrawn, or by incorporating one or more steps of washing the needle or tip after each sample is withdrawn. Alternatively, the connecting means can contact the capillary to be used for capillary electrophoresis directly with the amplification reaction in order to load an aliquot into the capillary.

In one embodiment, the first connecting means 66 transfers an aliquot of a PCR amplification reaction mixture from the amplification device to the analysis device at the end of each PCR cycle.

In another embodiment, the second connecting means 40 connects the polynucleotide extraction device with the amplification device. In another embodiment, the second connecting means also serves to replenish the amplification reaction mixture with a mixture comprising dNTPs, primers, necessary reagents, and a DNA polymerase at the same concentration as the starting reaction mixture. In still another embodiment, a different connecting means is used for replenishing the amplification reaction mixture. This connecting means may be made in the same way as described in this application to allow the transfer of fluid.

Preferably, the first connecting means of the present invention permits the feeding of an aliquot of the amplification reaction mixture into the analysis device, e.g., a capillary electrophoresis device. Such feeding function may be achieved by following known methods in the art, for example, as disclosed in U.S. Pat. Nos. 6,280,589; 6,192,768; 6,190,521; 6,132,582; and 6,033,546, all of which incorporated hereby by reference in their entireties.

In one embodiment, the sample is injected as a sample plug into a connecting means which comprises at least a channel for the electrolyte buffer and a supply and drain channel for the sample. The supply and drain channels discharge into the electrolyte channel at respective supply and drain ports of the analysis device 68. The distance between the supply port and the drain port geometrically defines a sample volume. The injection of the sample plug into the electrolyte channel is accomplished electrokinetically by applying an electric field across the supply and drain channels for a time at least long enough that the sample component having the lowest electrophoretic mobility is contained within the geometrically defined volume. The supply and drain channels each are inclined to the electrolyte channel. Means are provided for electrokinetically injecting the sample into the sample volume. The resistance to flow of the source and drain channels with respect to the electrolyte buffer is at least about 5% lower than the respective resistance to flow of the electrolyte channel.

In another embodiment, the sample is introduced by the first connecting means using the hydrodynamic method known in the art. The sample is injected into the capillary by a pressure difference. The pressure difference is produced either by placing the capillary ends at different levels, whereby a hydrostatic pressure difference is produced, or in a sealable sample reservoir overpressure is generated by means of gas, the overpressure injecting the sample solution into the capillary. The amount of sample passing into the capillary is controlled by the selection of the pressure difference and its effective time.

In another embodiment, the sample is injected by means of a fixed or movable sample-injection capillary by placing the sample-injection capillary in the vicinity of the inlet end of the capillary of the capillary zone electrophoresis apparatus in such a manner that the sample solution will surround the inlet end entirely, and sample is transferred into the separation capillary by means of an electrophoresis electric current or in some other manner, and after a predetermined time the solution is withdrawn from the vicinity of the inlet end, where the sample solution is replaced by the background solution.

In a different embodiment, however, no first connecting means is used to connect the amplification device with a capillary electrophoresis analysis device. Instead, a fraction of the amplified polynucleotide sample is loaded onto the electrophoresis device by directly immersing the capillaries and the electrodes of the electrophoresis device into PCR reaction. Preferably, an electric current may be applied to the electrodes for a limited time to force the polynucleotide sample to enter the capillaries by electrokinetic force as described above. The time to apply the electric current, for example, about 0.001 seconds, 0.01 seconds, 0.1 seconds, 1 seconds or 10 seconds or more, depends on the volume of samples need to be taken by the capillaries for the analysis by the capillary electrophoresis. This embodiment provides a simpler process for sample loading onto the analysis device.

The third connecting means 80 connects the analysis device 68 with a data generating device 120 which is located outside of the analysis device.

The fourth connecting means 140 is used in one embodiment to connect the analysis device 68 with the fraction collector 160. However, in another embodiment of the invention, no connecting means is used between the analysis device and the fraction collector device.

The fifth connecting means 180 is used in some embodiments to connect the sequence identifier 200 with the analysis device 68 or the fraction collector 160 so that the sequence identity of a polynucleotide of interest may be obtained.

In some embodiments, the first, second, fourth and fifth connecting means may be a single connecting means, for example, a robotic arm which permits fluids to transfer from one device to another device. The single robotic arm transfers fluids from one device to a second device, and then washes and cleans itself before it transfers fluids from one device to a third device.

Suitable substrates useful for making the connecting means of the invention may be fabricated from any one of a variety of materials, or combinations of materials. Often, the connecting means are manufactured using solid substrates commonly known in the art, e.g., silica-based substrates, such as glass, quartz, silicon or polysilicon, as well as other known substrates, i.e., gallium arsenide. Alternatively, polymeric substrate materials may be used to make the connecting means of the present invention, including, e.g., polydimethylsiloxanes (PDMS), polymethylmethacrylate (PMMA), polyurethane, polyvinylchloride (PVC), polystyrene polysulfone, polycarbonate, polymethylpentene, polypropylene, polyethylene, polyvinylidine fluoride, ABS (acrylonitrile-butadiene-styrene copolymer), and the similar materials.

The present invention permits an automated apparatus to be used for transcriptional profiling. The apparatus permits the amplification of a target polynucleotide and the quantitative analysis of the amplified products from the target polynucleotide. The apparatus may also permits polynucleotide extraction and reverse transcription. The apparatus may further permits the identification of a polynucleotide of interest (e.g., a gene that is differentially expressed in two or more samples), as well as the sequence identity of the polynucleotide of the interest.

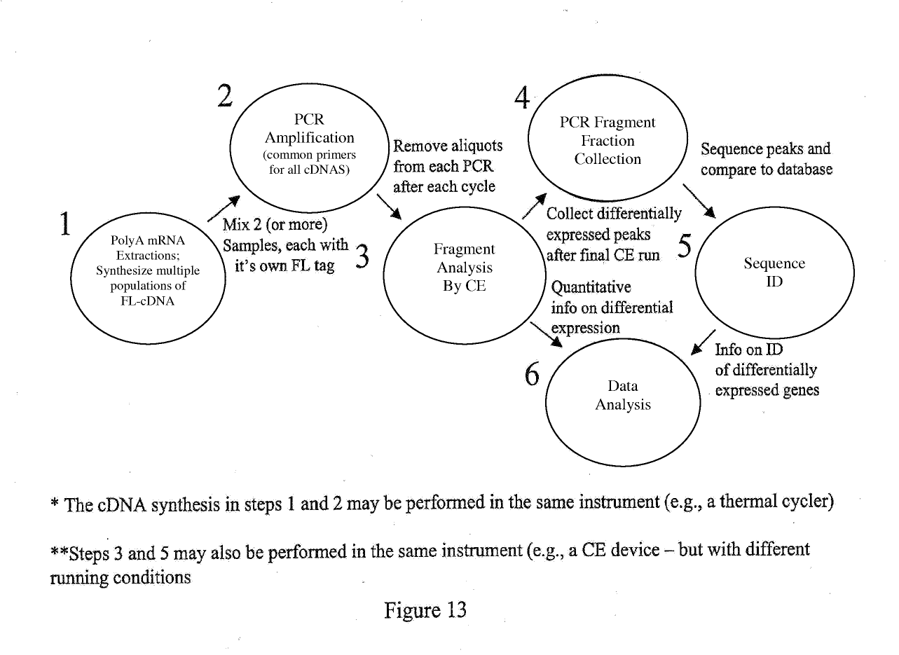

FIG. 13 demonstrates a schematic view of an expression profiling process using the apparatus of the present invention. For example, this process may be performed using the apparatus shown in FIG. 10. In a preferred embodiment, all devices in FIG. 10 are located within a single housing. RNAs may be extracted separately or may be extracted (step 1) by a polynucleotide extraction device connected to the amplification device as shown in FIG. 2. The amplification device 64 permits cDNA synthesis and amplification (e.g., by PCR, step 2). At the end of each PCR cycle, an aliquot of amplified product is removed to be analyzed on an analysis device 68 (e.g., a capillary electrophoresis device, step 3). Differentially expressed polynucleotides may be collected by a fraction collector 160 (step 4), and the sequence of one or more differentially expressed polynucleotides may be identified by a sequence identifier 200 (step 5). For step 5, a sequencing reagent master mix may be added and the sequencing reaction mixture may be incubated according to known methods in the art. In one embodiment, the sequencing reaction mixture is then loaded on to the analysis device 68 (e.g., the capillary electrophoresis device) for sequence identification. In another embodiment, an aliquot of a fraction collected by the fraction collector 160 may be returned to the amplification device 64 for performing sequence reaction. The reaction product may then be applied to the analysis device 68 for sequence identification.

In one embodiment, the apparatus of the present invention is used to analyze genomic DNA samples (e.g., quantitation of genomic copies of a gene). Such a technique would have lower cost and higher resolution than probe based assays or karyotyping, on a whole genome basis. The process for genomic DNA analysis may be performed similarly to the process for RNA analysis (e.g., as described above) except that there would be no need for reverse transcription and cDNA synthesis when genomic DNA is used as. The process for genomic DNA analysis may start with isolating genomic DNA from 2 or more samples to be compared. The samples may be split into multiple aliquots (e.g., 5, 10, 20, or 30 or more aliquots). Each aliquot may be amplified by a different primer set (e.g., 5, 10, 20, or 30 or more primer sets total for all aliquots to be analyzed). For each primer set, one primer could be complementary to a common repetitive sequence, or just a random sequence, and have a sample-specific sequence tag on it to make it sample-specific. The other primer could be a random primer. In one embodiment, the two or more samples are amplified under sane conditions, with same primers, then a ladder of PCR products would be formed that come from loci spread randomly throughout the genome. The quantities of each PCR product is then measured and compared between samples. Genome-wide differences in copy number at different loci can thus be identified. These differences are indicative of local duplications or amplifications; trisomy; and loss of heterozygosity.

Alternatively, a locus specific primer set (i.e., primers which recognize specific sequences at a target locus) may be used for PCR amplification for the determination of copy number changes at a specific locus between two or more samples.

The foregoing embodiments demonstrate experiments performed and techniques contemplated by the present inventors in making and carrying out the invention. It is believed that these embodiments include a disclosure of techniques which serve to both apprise the art of the practice of the invention and to demonstrate its usefulness. It will be appreciated by those of skill in the art that the techniques and embodiments disclosed herein are preferred embodiments only that in general numerous equivalent methods and techniques may be employed to achieve the same result.

All of the references identified hereinabove, are hereby expressly incorporated by reference in their entirety.

1. An automated modular apparatus for nucleic acid analysis, the apparatus comprising:
a PCR amplification device comprising a thermal cycler that performs PCR amplification cycles which amplify polynucleotides in a reaction mixture comprising a biological sample to generate an amplified polynucleotide product therein;
a capillary electrophoresis system comprising a capillary electrophoresis device and a detector, wherein said detector is arranged such that it detects nucleic acid species electrophoretically separated in a capillary electrophoresis capillary of said capillary electrophoresis device; and
a transport mechanism comprising a motorized stage that permits both horizontal and vertical movements for the purpose of transferring PCR reaction samples between the thermal cycler and the capillary electrophoresis system, the transport mechanism providing transient physical and electrical contact between a reaction mixture in said PCR amplification device and said capillary electrophoresis device to transfer a sample of a said reaction mixture from said amplification device to said analysis device during an amplification reaction, said capillary electrophoresis device comprising an electrode arranged to permit transient immersion of said electrode in a said reaction mixture,
wherein said transport mechanism permits the transient immersion of a said electrode and an end of a capillary electrophoresis capillary containing an electrophoretic separation medium into a said reaction mixture in said PCR amplification device during an amplification reaction to electrokinetically transfer a said sample of a said reaction mixture from said amplification device to said capillary electrophoresis device during an amplification reaction, such that said modular apparatus can automatically amplify, separate and detect a nucleic acid product in a said biological sample introduced to said PCR amplification device.

2. The automated modular apparatus in claim 1, further comprising a polynucleotide extraction device connected to said PCR amplification device which permits an extracted polynucleotide sample to transfer from said polynucleotide extraction device to said PCR amplification device.

3. The automated modular apparatus of claim 1, further comprising a fraction collector device.

4. The automated modular apparatus of claim 3, wherein said fraction collector device is connected to said capillary electrophoresis device so as to permit the collection of a quantified product.

5. The automated modular apparatus of claim 1, wherein said transport mechanism permits an aliquot of said reaction mixture to transfer from said amplification device to said capillary electrophoresis device at the end of each PCR cycle.

6. The automated modular apparatus of claim 1, wherein said apparatus permits the detection and quantification of a signal generated by one or more fluorescent labels.

7. A PCR system, the system comprising:
a thermal cycler performing PCR, receiving a raw DNA sample to be amplified into a DNA sample by PCR, in a form suitable for subsequent analysis;
a capillary electrophoresis system, receiving the DNA sample from the thermal cycler to be subject to capillary electrophoresis to analyze a result of the PCR;
and a modular system containing a transport mechanism that moves a stage in the horizontal and vertical directions for the purpose of transferring samples between the thermal cycler and the capillary electrophoresis system, wherein the capillary electrophoresis system and the thermal cycler are operative coupled within an automated modular system, whereby DNA sample output from the thermal cycler is input to the capillary electrophoresis system without further user intervention.

8. The PCR system of claim 7, wherein said transport mechanism permits an aliquot of said reaction mixture to transfer from said thermal cycler to said capillary electrophoresis system at the end of each PCR cycle.

9. The PCR system of claim 7, wherein said apparatus permits the detection and quantification of a signal generated by one or more fluorescent labels.

10. A PCR system, comprising:
a thermal cycler performing PCR, receiving a raw DNA sample to be amplified into a DNA sample by PCR, in a form suitable for subsequent analysis;
a capillary electrophoresis system, receiving the DNA sample from the thermal cycler to be subject to capillary electrophoresis to analyze result of the PCR; and
an integrated transport mechanism positioning the thermal cycler to be accessible by the capillary electrophoresis system, wherein the capillary electrophoresis system and the thermal cycler are operative coupled within an automated integrated system, whereby DNA sample output from the thermal cycler is input to the capillary electrophoresis system without further user intervention.

11. The PCR system of claim 10, wherein said transport mechanism permits an aliquot of said reaction mixture to transfer from said thermal cycler to said capillary electrophoresis system at the end of each PCR cycle.

12. The PCR system of claim 11, wherein said apparatus permits the detection and quantification of a signal generated by one or more fluorescent labels.

13. A bioanalysis system, comprising:
a sample preparation device, receiving a raw sample to be processed into a sample in a form suitable for subsequent analysis;
a sample analysis device, receiving the sample from the sample preparation device to be subject to analysis; and
an integrated transport mechanism positioning the sample preparation device to be accessible by the sample analysis device, wherein the sample preparation device and the sample analysis device are operative coupled within an integrated automated system, and wherein the raw sample is loaded into the sample preparation device, whereby sample output from the sample preparation device is input to the sample analysis device without further user intervention.
